(19) United States
(12) Patent Application Publication
(10) Pub. No.: US 2022/0087507 A1
(43) Pub. Date: Mar. 24, 2022

(54) IMAGING RECONSTRUCTION USING REAL-TIME SIGNAL OF ROTARY POSITION FROM NEAR DISTAL END ENCODER

(71) Applicant: Canon U.S.A., Inc., Melville, NY (US)

(72) Inventors: Ray Yan, Newton, MA (US); Alexander Altshuler, Cambridge, MA (US)

(21) Appl. No.: 17/424,844

(22) PCT Filed: Jan. 28, 2020
(86) PCT No.: PCT/US20/15486

§ 371 Date: Jul. 21, 2021

Related U.S. Application Data
(60) Provisional application No. 62/798,360, filed on Jan. 29, 2019.

(51) Int. Cl. A61B 1/00; A61B 5/00; A61B 8/00; A61B 8/12

(52) U.S. Cl. CPC A61B 1/00096; A61B 8/12; A61B 8/445; A61B 5/0062

(57) ABSTRACT

One or more devices, systems, methods, and storage mediums for imaging and for minimally invasive medical devices, such as, but not limited to, for intravascular ultrasound (IVUS), spectrally encoded endoscopy (SEE), and/or Optical Coherence Tomography (OCT), are provided herein. One or more embodiments may involve imaging reconstruction using a real-time signal of a rotary position from a near distal end encoder. One or more devices, systems, methods and storage mediums may include, in one or more embodiments, a rotary encoder or sensor to detect an angular position of a rotary shaft or a drive cable, for example, to reduce imaging Non-Uniform Rotational Distortion. Examples of such applications include imaging, evaluating and diagnosing biological objects, such as, but not limited to, for Gastro-intestinal, cardio and/or ophthalmic applications, and being obtained via one or more optical instruments.

### CROSS-REFERENCE TO RELATED APPLICATION(S)

This application relates, and claims priority, to U.S. Prov. Patent Application Ser. No. 62/798,360, filed Jan. 29, 2019, the disclosure of which is incorporated by reference herein in its entirety.

### FIELD OF THE INVENTION

The present disclosure relates generally to the field of imaging and more particularly to minimally invasive medical devices, such as, but not limited to, intravascular ultrasound (IVUS), spectrally encoded endoscopy (SEE) and/or Optical Coherence Tomography (OCT) apparatuses and systems, and methods and storage mediums for use with same. Examples of SEE applications include imaging, evaluating and characterizing/identifying biological objects or tissue, such as, but not limited to, for gastro-intestinal, cardio and/or ophthalmic applications. Examples of OCT applications include imaging, evaluating and diagnosing biological objects, such as, but not limited to, for gastro-intestinal, cardio and/or ophthalmic applications, and being obtained via one or more optical instruments, such as, but not limited to, one or more optical probes, one or more catheters, one or more endoscopes, one or more capsules, and one or more needles (e.g., a biopsy needle). One or more devices, systems, methods and storage mediums may be for characterizing, examining and/or diagnosing, a sample or object in application(s) using an apparatus or system that includes, in one or more embodiments, a flexible rotary shaft or drive cable, and in one or more other embodiments, a driving motor, a rotary encoder, a probe, a first drive cable connecting the driving motor to the rotary encoder, and a second drive cable connecting the rotary encoder to the probe. In one or more embodiments, use of a rotary encoder or sensor to detect an angular position of a flexible rotary shaft or drive cable, for example, may reduce imaging Non-Uniform Rotational Distortion (NURD).

### BACKGROUND OF THE INVENTION

Spectrally encoded endoscope (SEE) is an endoscope technology which uses a broadband light source, a rotating grating and a spectroscopic detector to encode spatial information on a sample. When illuminating light to the sample, the light is spectrally dispersed along one illumination line, such that the dispersed light illuminates a specific position of the illumination line with a specific wavelength. When the reflected light from the sample is detected with the spectrometer, the intensity distribution is analyzed as the reflectance along the line. By rotating or swinging the grating back and forth to scan the illumination line, a two-dimensional image of the sample is obtained.

Optical coherence tomography (OCT) is a technique for obtaining high resolution cross-sectional images of tissues or materials, and enables real time visualization. The aim of the OCT techniques is to measure the time delay of light by using an interference optical system or interferometry, such as via Fourier Transform or Michelson interferometers. A light from a light source delivers and splits into a reference arm and a sample (or measurement) arm with a splitter (e.g., a beamsplitter). A reference beam is reflected from a reference mirror (partially reflecting or other reflecting element) in the reference arm while a sample beam is reflected or scattered from a sample in the sample arm. Both beams combine (or are recombined) at the splitter and generate interference patterns. The output of the interferometer is detected with one or more detectors, such as, but not limited to, photodiodes or multi-array cameras, in one or more devices, such as, but not limited to, a spectrometer (e.g., a Fourier Transform infrared spectrometer). The interference patterns are generated when the path length of the sample arm matches that of the reference arm to within the coherence length of the light source. By evaluating the output beam, a spectrum of an input radiation may be derived as a function of frequency. The frequency of the interference patterns corresponds to the distance between the sample arm and the reference arm. The higher frequencies are, the more the path length differences are. Single mode fibers may be used for OCT optical probes, and double clad fibers may be used for fluorescence and/or spectroscopy.

Using optical fiber for imaging is getting more and more prevalent in a number of applications that may benefit from small probe size and high fidelity images. In most of these applications in order to provide reasonable field of view it is useful to rotate and/or longitudinally translate the fiber. Such rotation and/or translation of the fiber usually leads to a relatively bulky mechanism comprising an optical Rotary Junction (RJ), a rotational motor, and, sometimes, a linear stage. From one side, for the ease of use it is preferable to keep a Probe Interface Unit (PIU) inside a main system console. On the other side, for a flexible probe the rotational motion is usually imparted to the optical fiber by a flexible drive shaft disposed inside the sheath of the probe. Though this drive shaft is designed to be torsionally rigid it is still experiencing uneven wind-up in rotation leading to a Non-Uniform Rotational Distortion (NURD) compromising the final image.

Many of such high resolution imaging means (e.g., IVUS, OCT, SEE, etc.) are dependent on the use of a flexible rotary shaft (also called a drive cable or drive-shaft) to transmit torque to an imaging device at the distal end. When a flexible drive-shaft transmits the scan rotation from a proximal motor to the distal end probe, rotation transmission may not be entirely one to one. If only the proximal rotation is used as a reference to form a scan image, the rotational NURD error distorts the image. The nature of using a flexible rotary shaft/drive cable to transmit torque from a proximal motor to a distal imaging device constantly causes the rotational NURD error, which distorts the image. The image may be rendered useless or may cause clinical misjudgments when distortions occur in a medical scope or imaging device. Current attempts to mitigate the NURD issue require additional physical means of the imaging probes and have many constraints and disadvantages, which include, but are not limited to, increased complexity, high cost, low resolution, and low accuracy.

Accordingly, it would be desirable to provide at least one imaging (e.g., SEE, IVUS, OCT, etc.) technique, storage medium and/or apparatus or system for use in at least one optical device, assembly or system to achieve efficient characterization and/or identification of biological object(s) or tissue, especially in a way that reduces or minimizes cost of manufacture and maintenance and/or in a way that reduces or eliminates NURD.

### SUMMARY OF THE INVENTION

Accordingly, it is a broad object of the present disclosure to provide imaging (e.g., SEE, IVUS, OCT (for example, but not limited to, using an interference optical system, such as an interferometer (e.g., spectral-domain OCT (SD-OCT), swept-source OCT (SS-OCT), multimodal OCT (MM-OCT), etc.)), etc.) apparatuses and systems (e.g., using a flexible rotary shaft or drive cable), and methods and storage mediums for use with same. It is also a broad object of the present disclosure to provide OCT devices, systems, methods, and storage mediums using a flexible rotary shaft or drive cable.

It is also a broad object of the present disclosure to provide imaging apparatuses, systems, methods and storage mediums to the field of minimally invasive medical imaging devices, including, but not limited to, intravascular ultrasound (IVUS) (or any other intravascular imaging (WI) modality or modalities), optical coherence tomography (OCT), and Spectrally Encoded Endoscopy (SEE). One or more embodiments of the present disclosure utilize a rotary encoder/sensor to detect the angular position of a rotary shaft or a drive cable. The rotary encoder or sensor may be placed between the proximal driving motor and the distal imaging probe in one or more embodiments. The real-time digital signal of the angular position of the drive cable may be acquired simultaneously with the scanned imaging signal from the probe by imaging software for image construction or reconstruction (e.g., in accordance with one or more methods discussed herein, using one or more processors discussed herein, etc.). By using the configuration(s) and technique(s) discussed herein, NURD may be reduced and/or eliminated (from the reconstructed images) from the proximal motor end up to where the rotary encoder is placed such that the reconstructed images are not distorted by the NURD.

In one or more embodiments, a computer-readable storage medium may store at least one program that operates to cause one or more processors to execute a method for performing image reconstruction (e.g., with an encoder), where the method may include one or more steps discussed herein.

In one or more embodiments, more precise or more accurate estimate of a true rotary position of a distal rotary shaft or an imaging assembling attached to the rotary shaft may help overcome distortion (e.g., NURD) by providing more accurate information for image reconstruction. In one or more examples, an angular position or rotary speed sensor, such as an optical rotary encoder, may be placed between a proximal driving motor and a distal end of an imaging probe or scope near a point from which the imaging beam is directed towards a target or targets. Real-time rotary scanning position data output from the rotary encoder may be used by a computer or processor (e.g., via imaging software) for scanning image construction/reconstruction.

In one or more embodiments, a real-time digital signal of the shaft (or drive cable) or an angular position of the drive cable or cables may be acquired (e.g., simultaneously) with the imaging signal from the probe or scope by a computer or processor (e.g., via imaging software) for image construction or reconstruction. NURD error(s) generated by the portion of the drive cable between the proximal motor and the rotary encoder may be eliminated from the constructed or reconstructed images. In one or more embodiments, two drive cables (or shafts) may be used for proximal and distal sides of the encoder, respectively. In one or more embodiments, connection couplers may be used between a first drive cable and the rotary encoder and between the rotary encoder and a second drive cable. In one or more embodiments, the first drive cable, the second drive cable, and/or the rotary encoder may be either disposable or reusable independently.

In one or more embodiments, one rotary encoder may be used near a distal end of a rotary probe or scope. A portion of the probe with the encoder or sensor integrated may be equal or much larger outer diameter (OD) than a rest (or remaining portion) of the probe/scope OD, which may provide sufficient space for the integration of the rotary encoder or sensor.

In accordance with one or more embodiments of the present disclosure, apparatuses and systems, and methods and storage mediums for performing image reconstruction may further operate to characterize biological objects, such as, but not limited to, blood, mucus, tissue, etc.

In one or more embodiments, an imaging system may include: a probe having a proximal end and a distal end operating to communicate a probing signal with a specimen, object, or target; at least one drive cable; and a rotary encoder or sensor in communication with, or attached to, the at least one drive cable, the probe being positioned on a first side or a distal side of the rotary encoder or sensor, wherein the probe, the at least one drive cable, and the rotary encoder or sensor rotate using a drive, and wherein a first portion of, or a first drive cable of, the at least one drive cable is positioned on a second side or a proximal side of the rotary encoder or sensor, and has a length such that non-uniform rotational distortion (NURD) distortion(s) is/are reduced, minimized, and/or eliminated from the image of the specimen, object, or target.

In one or more embodiments, the rotary encoder or sensor of the probe one or more of: (i) includes, or is connected to, optical fiber; (ii) is disposed between the first portion of, or the first drive cable, of the at least one drive cable and a second portion of, or a second drive cable of, the at least one drive cable; (iii) is disposed such that the a first portion of, or a first drive cable of, the at least one drive cable is positioned on the second side or the proximal side of the rotary encoder or sensor and the second portion of, or the second drive cable of, the at least one drive cable is located on the first side or the distal side of the rotary encoder or sensor; (iv) is disposed such that the a first portion of, or a first drive cable of, the at least one drive cable is positioned on the second side or the proximal side of the rotary encoder or sensor and the second portion of, or the second drive cable of, the at least one drive cable is located on the first side or the distal side of the rotary encoder or sensor such that the second portion of, or the second drive cable of, the at least one drive cable is located between the rotary encoder or sensor and the probe; (v) is disposed on one end of the second portion of, or the second drive cable of, the at least one drive cable and the probe is located on another end of the second portion of, or the second drive cable of, the at least one drive cable; (vi) has the at least one drive cable extending through the rotary encoder or sensor; and/or (vii) operates to measure an angular position of the at least one drive cable, the first portion of the at least one drive cable and the second portion of the at least one drive cable, or the first drive cable of the at least one drive cable and the second drive cable of the at least one drive cable.

One or more embodiments may include first and second connection couplers, the first connection coupler being disposed between the first portion of, or the first drive cable of, the at least one drive cable and the rotary encoder or sensor, and the second connection coupler being disposed between the second portion of, or the second drive cable of, the at least one drive cable and the rotary encoder or sensor. In one or more embodiments, one or more of the first drive cable or the first portion of the at least one drive cable, the second drive cable or the second portion of the at least one drive cable, and the rotary encoder or sensor may operate to be disposable or reusable independently.

One or more embodiments may include the drive that rotates the probe, the at least one drive cable, and the rotary encoder or sensor, wherein one or more of the following: (i) the drive is disposed such that the first portion of, or the first drive cable of, the at least one drive cable is positioned between the drive and the rotary encoder or sensor; (ii) the length of the first portion of, or the first drive cable of, the at least one drive is the length between the drive and the rotary encoder or sensor; and/or (iii) the drive is a proximal driving motor.

In one or more embodiments, one or more of the following may occur/exist: (i) data or a signal from the rotary encoder or sensor comprises real-time rotary or angular position data from the rotary encoder or sensor; (ii) the rotary encoder or sensor is used near a distal end of the probe or at a predetermined length or distance from the probe to reduce, minimize, or avoid/eliminate the NURD; and/or (iii) the rotary encoder or sensor is used near a distal end of the probe or at a predetermined length or distance from the probe to reduce, minimize, or avoid/eliminate the NURD, where the predetermined length or distance from the probe is in a range from about one centimeter to about 50 centimeters away from the distal end of the probe.

One or more embodiments may include a mass applied to the at least one drive cable at a location of the rotary encoder or sensor such that the mass operates as a flywheel to achieve an even rotation speed and/or to improve a rotational inertia of the rotational component(s) at the location of the mass, wherein the improved rotational inertia operates to stabilize the rotary motion and to reduce rotational speed variation(s) and the NURD.

In one or more embodiments, one or more of the following may occur: (i) the at least one drive cable comprises a single drive cable used or extending from the probe, wherein the single drive cable is disposed in and/or through a center hole or a hollow shaft of the rotary encoder or sensor and mechanically attaches to a code disk or wheel of the rotary encoder or sensor; and/or (ii) the system further comprises the drive, the the at least one drive cable comprises the single drive cable used or extending between the drive and the probe, and the single drive cable is disposed in and/or through the center hole or the hollow shaft of the rotary encoder or sensor such that the single drive cable mechanically attaches to a code disk or wheel of the rotary encoder or sensor. In a case where the single drive cable drives the probe, the rotary encoder or sensor or a portion thereof may rotate together with the single drive cable.

One or more embodiments may include one or more processors, where the one or more processors may operate to: (i) acquire the probing signal and/or data of the rotary encoder or sensor to reduce, minimize, and/or eliminate the NURD, and/or (ii) to generate or reconstruct the image having the reduced, minimized, and/or eliminated NURD. The one or more processors may receive the probing signal and/or the data simultaneously and/or in real-time, and reduce, minimize, or eliminate the NURD from the generated image or while generating or reconstructing the image.

In one or more embodiments, reducing or minimizing NURD means to reduce or minimize NURD as compared to an imaging system having a rotary encoder proximal to a drive or at a same location with the drive, where the length of the component of the imaging system that includes the drive cable(s) and the probe is the same as the length of the claimed imaging system component that includes the drive cable, the rotary encoder, any second drive cable, and the probe. In one or more embodiments, where the imaging system has a proximal rigid cable or tube, this rigid cable or tube is not included in the component of the imaging system such that the length of the rigid portion is not included in the comparison.

In one or more embodiments, wherein an integration portion of the probe to include the encoder or sensor integrated therein may have an equal or larger outer diameter (OD) than a rest, or a remaining portion, of the probe such that sufficient space is provided for the integration of the rotary encoder or sensor. In one or more embodiments, an apparatus or system may include one or more of the following: (i) a sheath or a handle in which the probe is located; and/or (ii) a stationary portion of the system, wherein the stationary portion comprises at least a signal source and one or more signal detector subsystems.

In one or more embodiments, an imaging system may include: a probe having a rotary encoder or sensor, a proximal end with a signal transmitting connector, and a distal end operating to communicate a probing signal with a specimen, object or target, wherein data from the rotary encoder or sensor and/or the probing signal are used by the imaging system to generate an image of the specimen, object or target, and wherein the rotary encoder or sensor operates to trace the position of the drive cable to reduce and/or eliminate non-uniform rotational distortion (NURD).

In accordance with one or more embodiments of the present disclosure, SEE apparatuses and systems, and methods and storage mediums may operate to characterize tissue type in addition to providing a morphological image to help an operator's diagnostic decision based on quantitative tissue information. In accordance with one or more embodiments of the present disclosure, SEE apparatuses and systems, and methods and storage mediums may operate to characterize biological objects other than tissue. For example, the characterization may be of a biological fluid such as blood or mucus.

In accordance with at least another aspect of the present disclosure, one or more technique(s) discussed herein may be employed to reduce the cost of at least one of manufacture and maintenance of the one or more apparatuses, devices, systems and storage mediums by reducing or minimizing a number of disposable components and by virtue of the efficient techniques to cut down cost of use/manufacture of such apparatuses, devices, systems, and storage mediums.

According to other aspects of the present disclosure, one or more additional devices, one or more systems, one or more methods, and one or more storage mediums using, or for use with, one or more image reconstruction techniques are discussed herein. Further features of the present disclosure will in part be understandable and will in part be apparent from the following description and with reference to the attached drawings.

### BRIEF DESCRIPTION OF THE DRAWINGS

For the purposes of illustrating various aspects of the disclosure, wherein like numerals indicate like elements, there are shown in the drawings simplified forms that may be employed, it being understood, however, that the disclosure is not limited by or to the precise arrangements and instrumentalities shown. To assist those of ordinary skill in the relevant art in making and using the subject matter hereof, reference is made to the appended drawings and figures, wherein:

FIG. 1 is a diagram showing an embodiment of an apparatus or system which may utilize an encoder, two drive cables, one or two drive cable couplers, and image construction or reconstruction technique(s) in accordance with one or more aspects of the present disclosure;

FIG. 2 is a diagram showing an embodiment of an apparatus or system which may utilize an encoder, one drive cable, and image construction or reconstruction technique(s) in accordance with one or more aspects of the present disclosure;

FIG. 3 is a diagram showing an embodiment of an apparatus or system which may utilize an encoder, two drive cables, one or two drive cable couplers, and image construction or reconstruction technique(s) in accordance with one or more aspects of the present disclosure;

FIGS. 4A-4B are diagrams showing at least two embodiments of an apparatus or system which may utilize disposable portion(s) located at different sections or extending along different lengths or portions in accordance with one or more aspects of the present disclosure;

FIG. 5 is a diagram of an embodiment of a catheter that may be used with at least one embodiment of an apparatus or system which may utilize one encoder and image construction or reconstruction techniques in accordance with one or more aspects of the present disclosure;

FIG. 6 is a diagram showing an embodiment of at least an apparatus or system may use an encoder or sensor in accordance with one or more aspects of the present disclosure;

FIG. 7 is a diagram showing at least one embodiment of an apparatus or system which operates to utilize a SEE technique with an encoder or a sensor for optical probe applications in accordance with one or more aspects of the present disclosure;

FIG. 8 shows a schematic diagram of an embodiment of a computer that may be used with one or more embodiments of at least one apparatus, system, method, and/or storage medium, including, but not limited to, for use with one encoder/sensor and for performing one or more image construction/reconstruction techniques, in accordance with one or more aspects of the present disclosure; and

FIG. 9 shows a schematic diagram of another embodiment of a computer that may be used with one or more embodiments of at least one apparatus, system, method, and/or storage medium, including, but not limited to, for use with one near distal end encoder/sensor and for performing one or more image construction/reconstruction techniques, in accordance with one or more aspects of the present disclosure.

### DETAILED DESCRIPTION OF THE INVENTION

One or more devices/apparatuses, optical systems, methods, and storage mediums for use with an encoder or sensor (e.g., a near distal end encoder or sensor) and for performing one or more image construction/reconstruction techniques are disclosed herein.

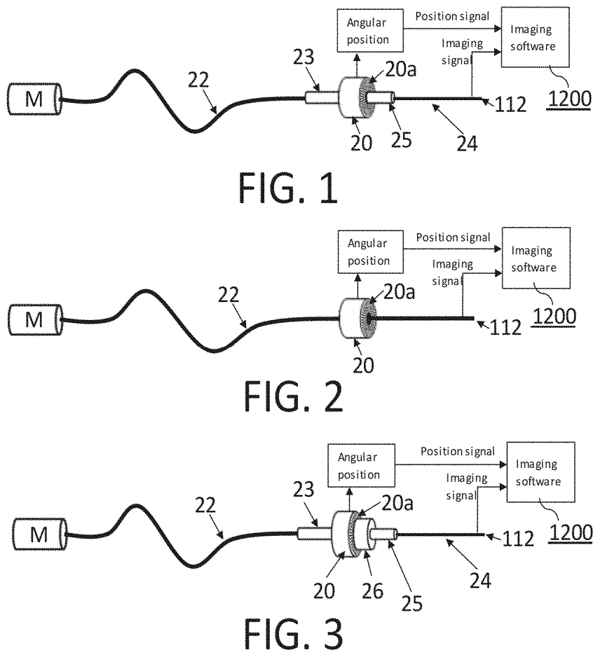

One or more systems, devices, methods and storage mediums are provided herein, including, but not limited to, the embodiment as shown in FIG. 1, which includes at least a Rotary Encoder 20, a Proximal Driving Motor M, and a probe or distal imaging probe 112 (also referred to as a scope or the scope 112). The Rotary Encoder 20 may be placed between the Proximal Driving Motor M and the distal imaging Probe 112. The code disk or wheel 20a (best seen in FIGS. 1-3) of the rotational encoder 20 may be connected to the driving motor M through the drive cable 1 (reference number 22 in FIG. 1) and drive cable coupler 1 (reference number 23 in FIG. 1) on one side and the rotary probe 112 through the other drive cable 2 (reference number 24 in FIG. 1) and drive cable coupler 2 (reference number 25 in FIG. 1) on the other side. In one or more embodiments, the encoder 20 may include a stator part. The stator part of the encoder 20 is preferably on the stationary part, which, in one or more embodiments, may be just a sheath or a handle. For example, rotary optical encoders typically comprise or include an LED light source, light detector, a code disk, and a signal processor. The light source, light detector, and signal processor, etc. are usually integrated on a printed circuit board (PCB) forming the stator part. In one or more embodiments, the encoder stator part may be mounted on the outer ring of a bearing within a handle as the stationary part. In addition, the code disk may be mounted on a shaft and supported by the inner ring of a bearing which is driven by the drive cable to make up the rotary part. In such an embodiment(s), the drive cable couplers 23 and 25 may attach drive cable 22 and 24 to the encoder shaft on both ends, respectively.

The Proximal Driving Motor M may transmit torque through the flexible Drive Cable-1 (reference number 22 in FIG. 1) to drive the Rotary part such as a coded disk 20a in the Rotary Encoder 20. At the same time, the torque on the Rotary Encoder 20 may be transmitted to the Drive Cable-2 (reference number 24 in FIG. 1) at the distal end (e.g., an end of the drive cable 24 that connects to the probe 112) to drive the probe 112 to rotate. The real-time digital signal of the angular position of the Drive Cable-1 (reference number 22 in FIG. 1) may be acquired simultaneously with the imaging signal from the Probe 112 by a processor or a computer (e.g., the computer 2, the processor or computer 1200, the processor or computer 1200′, any other processor discussed herein, etc.) as discussed further below, for example, via Imaging Software (e.g., in accordance with one or more methods discussed herein, using one or more processors or computers discussed herein, etc.). NURD issues may be avoided or eliminated (from the reconstructed images) from being caused by the flexible Drive Cable-1 (reference number 22 in FIG. 1) and the Proximal Motor M such that the reconstructed images are not distorted by the NURD. Other than rotary optical encoders, another example is a hall-effect magnetic encoder using a wheel attached to the rotary shaft to be tracked, and the wheel may be magnetized with north and south poles around its perimeter. In addition to using a bearing, in one or more embodiments, the stationary portion of the rotary encoder, such as the PCB board, may be attached to the rotary drive cable sheath by mechanical means, and the rotary disk or wheel may be attached to the rotary drive cable mechanically (e.g., through the one or two drive cable couplers).

As best seen in FIG. 2, in one or more additional embodiments, a single drive cable 22 may be used between the Proximal Driving Motor M and the distal Probe 112. In at least one embodiment, the Drive Cable 22 preferably goes through a center hole or hollow shaft (best seen in FIG. 2 showing the center hole or hollow shaft) of the Rotary Encoder 20 and mechanically attaches to the Encoder code disk or wheel 20a and/or the Probe 112. When this Drive Cable 22 drives the distal Probe 112, the Encoder disk 20a is rotating together with the Drive Cable 22 in one or more embodiments. The Rotary Encoder 20 may be placed in a location as close as possible (e.g., a predetermined location, a predetermined location in relation to the probe 112 or the distal end of the probe 112, a set distance or length from the distal end of the probe 112, a few centimeters from the distal end of the probe 112, anywhere in a range from about one centimeter or more to ₅o centimeters away from the distal end of the probe 112, etc.) to the Probe 112 to reduce, minimize, or avoid NURD in one or more embodiments. This single Drive Cable 22 may transmit the motor rotation to both the Rotary Encoder disk 20a and the imaging Probe 112. As similarly achieved using the aforementioned embodiment(s), the NURD issues that may arise or be caused between the Encoder 20 and the Drive Motor M may be eliminated from the constructed or reconstructed images.

As best seen in FIG. 3, one or more embodiments may use a cylindrical part 26 with a through hole at its center, the cylindrical part 26 being attached mechanically to the rotary drive cables, the rotary encoder shaft, or the drive cable couplers. The cylindrical part 26 preferably provides a certain amount of extra mass to the drive cables and the Rotary Encoder disk 20a to function as a “flywheel” (e.g., a device that mechanically is designed to store rotational energy, a device that may resist a change or changes in rotational speed by its moment of inertia, a device that may achieve a predetermined or desired rotational speed by its moment of inertia, etc.) to improve the rotational inertia of the rotational components at this location. In one or more embodiments, the cylindrical part 26 that functions as a flywheel may be disposed on either side (e.g., the proximal side, the distal side, etc.) of the rotary encoder or sensor 20. In one or more embodiments, the part 26 may have a different shape other than cylindrical (e.g., semi-circular, ovular, any other shape, etc.). The Mass component or cylindrical part 26 may be made by using, or of, the desired material with optimized mass and dimensions to achieve the desired rotational inertia depending on the driving torque, product design, and other requirements. With using this Mass component or cylindrical part 26, the improved rotational inertia may help stabilize the rotary motion and reduce the rotational speed variation and imaging NURD.

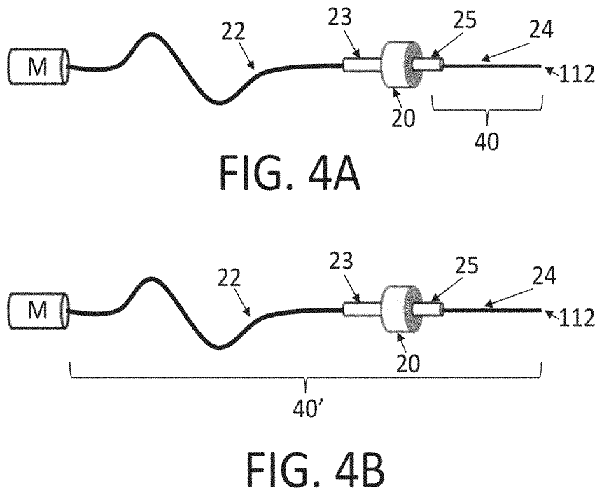

As shown in FIGS. 4A-4B, one or more embodiments may use different, disposable portions (e.g., disposable portion 40 in FIG. 4A, disposable portion 40′ in FIG. 4B, etc.) of imaging devices with two Drive Cables 22, 24 and a Rotary Encoder 20 in between the two Drive Cables 22, 24. Drive Cable-2 (reference number 24 as shown in FIGS. 4A-4B) may be connected to the Rotary Encoder 20 by the Drive Cable Coupler -2 (reference number 25) as shown in FIGS. 4A-4B. The distal portion of the imaging device may be disposed by disconnecting the Coupler-2 (reference number 25) from the Rotary Encoder 20 (see e.g., FIG. 4A), and the rest of the device (or other predetermined portions or combinations thereof) may be reusable. By way of at least one embodiment example, one, more than one, or all of the components between the Motor M and the distal end (e.g., where the probe or scope 112 is located) may be disposable, including, but not limited to, the Drive cable-1 (reference number 22), Encoder 20, Drive cable-2 (reference number 24), Couplers 23 and 25, and the probe or scope 112 (see e.g., FIG. 4B).

By using a rotary encoder 20 near the distal end of the imaging probe 112, in one or more embodiments, the angular position of the drive cable (e.g., the drive cable 22, the drive cable 24, both drive cables 22, 24, etc.) may be acquired in real-time with the imaging signal from the probe 112. As aforementioned, the NURD issues may be eliminated or avoided that would otherwise occur from the proximal end (e.g., where the motor M is located, near the motor M, near or at the end of the drive 22 that is connected to the motor M, etc.) up to where the rotary encoder 20 is placed, and the constructed or reconstructed images may avoid such distortion.

The Rotary Encoder or sensor 20 for the angular position of the drive cable may be placed within or using, or from a certain distance (or predetermined distance) from or of, the probe 112 tip. Such placement may eliminate a majority of the NURD for a long rotational driving path, but at the same time, such configurations avoid space limitation(s) related to installing an encoder/sensor 20 inside a distal tip of the probe 112. The subject configurations provide the advantage of using a higher resolution, lower cost, and less complex encoder for the positioning signal.

The high rotational inertia from the rotary encoder 20 and/or the Mass or cylindrical component 26 may improve the rotation uniformity of the probe 112 and further reduce the imaging NURD in one or more embodiments.

The two independent Drive Cables 1 and 2 (e.g., the drive cable 22 and the drive cable 24, respectively) may provide the flexibility of different disposable portion(s) for lowering cost.

In torque and axial force transmission applications, drive shafts are usually enclosed in stationary close fitting non-rotating sheaths to provide for rotational support, safety, and to facilitate axial motion of the shaft or shafts. These sheaths are preferably made of, or internally lined by, a low friction material, such as polytetrafluoroethylene (PTFE). Such sheaths may be used in one or more embodiments.

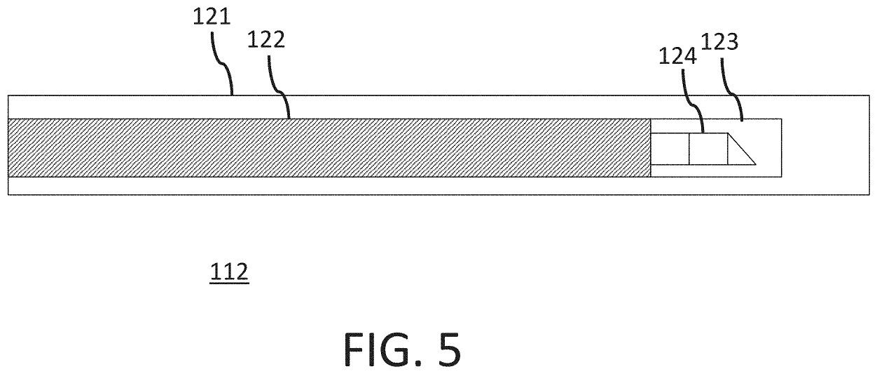

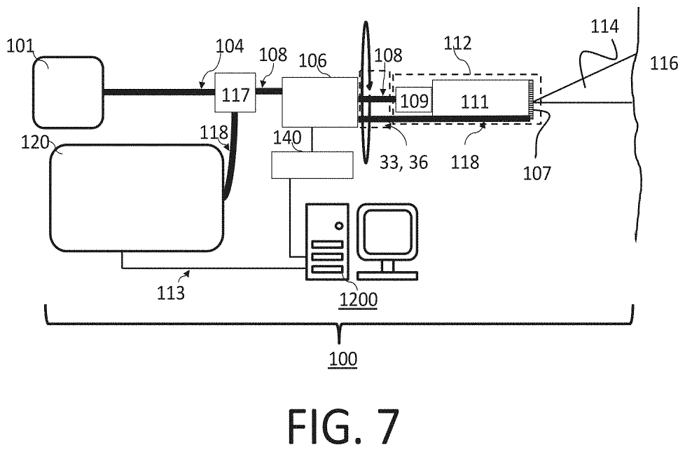

In one or more embodiments, a probe and/or scope (e.g., such as, but not limited to, the probes and/or scopes as shown diagrammatically in FIGS. 1-4B) may be an embodiment of the catheter 112 (e.g., as shown in FIG. 5) including a sheath 121, a coil 122, a protector 123 and an optical probe 124. The catheter 112 may be connected to a probe interface unit (PIU) as aforementioned, or to a patient interface unit, to spin the coil 122 with pullback (e.g., at least one embodiment operates to spin the coil 122 with pullback). The coil 122 delivers torque from a proximal end to a distal end thereof (e.g., via or by a motor, such as, but not limited to, a proximal driving motor as aforementioned). In one or more embodiments, the coil 122 is fixed with/to the optical probe 124 so that a distal tip of the optical probe 124 also spins to see an omnidirectional view of a biological organ, sample or material being evaluated, such as, but not limited to, hollow organs such as vessels, a heart, etc. For example, fiber optic catheters and endoscopes may reside in a sample arm of an OCT interferometer, or in a probe/catheter of an SEE apparatus/system, in order to provide access to internal organs, such as intravascular images, gastro-intestinal tract or any other narrow area, that are difficult to access. As the beam of light through the optical probe 124 inside of the catheter 112 or endoscope is rotated across the surface of interest, cross-sectional images of one or more samples are obtained. In order to acquire three-dimensional data, the optical probe 124 is simultaneously translated longitudinally during the rotational spin resulting in a helical scanning pattern. This translation may be performed by pulling the tip of the probe 124 back towards the proximal end and therefore referred to as a pullback. As discussed further below, the scope or probe may have different structure in one or more embodiment, such as, but not limited to, structure that is the same as or similar to the probe 112 shown in FIG. 7.

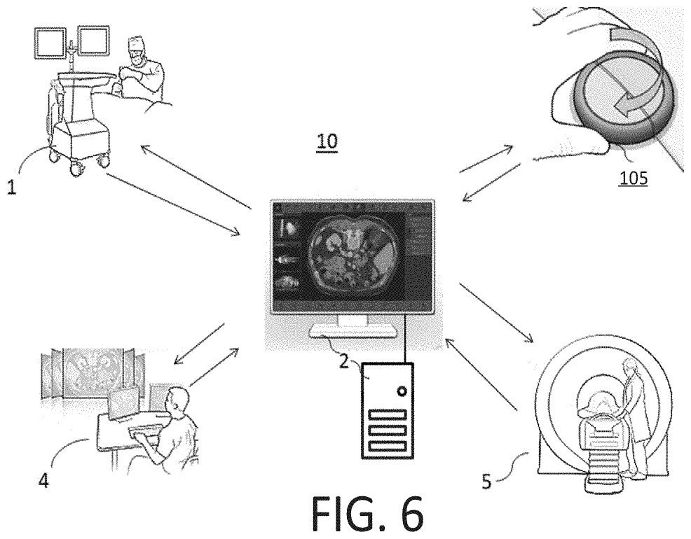

As shown in FIG. 6, at least one embodiment of an apparatus or system may use an encoder or sensor in accordance with one or more aspects of the present disclosure. For example, a medical device 1, 105, 5, etc. may be used with one or more of the encoder or sensor apparatuses or systems discussed herein. For example, the system 2 may communicate with the image scanner 5 to request information for use in a medical imaging procedure (e.g., in a needle guidance planning and/or performance procedure, an SEE procedure, an OCT procedure, an IVUS procedure, etc.), such as, but not limited to, bed or slice positions, and the image scanner 5 may send the requested information along with the images to the system 2 once a clinician uses the image scanner 5 to obtain the information via scans of the patient. By way of another example, the system 2 may communicate and be used with a guidance device (also referred to as a locator device) 105 (such as an image-plane localizer that may be a patient-mount device and may be rotated as shown to help locate to biological object, such as a lesion or tumor). The aforementioned embodiments shown in FIGS. 1-5 may be employed in or with the system 10, the computer or system 2, the medical device 1, the device 105, the scanner 5, etc., to obtain information from the patient when conducting medical procedures. The system 2 may further communicate with a PACS 4 to send and receive images of a patient to facilitate and aid in the medical procedure planning and/or performance. Once the plan is formed, a clinician may use the system 2 along with a medical device (e.g., a medical imaging device using the encoder or sensor technology discussed herein, a biopsy device using the encoder or sensor technology discussed herein, an ablation device using the encoder or sensor technology herein, an OCT device using the encoder or sensor technology herein, an SEE device using the encoder or sensor technology herein, an IVUS device using the encoder or sensor technology herein, etc.) 1 to consult a chart or plan (e.g., for needle guidance, for ablation, for biopsy, for imaging, for a medical procedure, etc.) to understand the shape and/or size of the targeted biological object to undergo the medical procedure (e.g., ablation, biopsy, imaging, etc.). Each of the medical device 1, the system 2, the guidance device 105, the PACS 4 and the scanning device 5 may communicate in any way known to those skilled in the art, including, but not limited to, directly (via a communication network) or indirectly (via one or more of the other devices 1, 105 or 5; via one or more of the PACS 4 and the system 2; via clinician interaction; etc.). In one or more embodiments as discussed herein, the guidance device 105 may communicate wirelessly with one or more of the following: the medical device 1, the system 2, the PACS 4, and the scanning device 5. Preferably, in one or more embodiments, the guidance device 105 communicates wirelessly with at least the system 2 or any other processor operating to interact with the guidance device 105 to perform the procedure(s) discussed herein.

One or more embodiment examples of apparatuses or systems may use the encoder or sensor features discussed herein. By way of at least one non-limiting, non-exhaustive embodiment example, FIG. 7 shows a (“SEE”) system 100 (also referred to herein as “system 100” or “the system 100”) which operates to utilize a SEE technique with an encoder and/or a sensor for optical probe applications in accordance with one or more aspects of the present disclosure. As shown in FIG. 7, light emitted by a white light source 101 is transmitted by at least one illumination light transmission fiber 104 and/or 108 and is incident on a probe portion 112 (also referred to herein as “probe section 112” or “the probe 112”) via a rotary junction (hereinafter, RJ) 106 (e.g., the at least one fiber(s) 104 and/or 108 may extend through the RJ 106 and into the probe portion 112) and/or may be incident on a probe portion (e.g., the probe portion 112) via the Rotary Encoder or Sensor 33, 36 (in one or more embodiments, the rotary encoder or sensor 33, 36 may have the same or similar features as shown for the numerous non-exhaustive, non-limiting embodiment examples of the rotary encoder or sensor 20 such that the aforementioned features of the encoder or sensor 20 are not repeated with reference to FIG. 7). Additionally or alternatively, the light emitted by the white light source 101 may be transmitted by the at least one illumination light transmission fiber 104, 108 and is incident on the probe 112 via a deflecting or deflected section 117 and via the RJ 106 as shown in FIG. 7, for example. In one or more embodiments (see e.g., as discussed in at least U.S. patent application Ser. No. 16/184,832, filed Nov. 8, 2018, the disclosure of which is incorporated by reference herein in its entirety) a length of an optical fiber 108 (e.g., a first stationary fiber) may extend from the deflecting or deflected section 117 and connect to one side of the RJ 106, and a length of a different optical fiber 108 (e.g., that operates to receive light that is coupled from the RJ 106 to the different optical fiber 108 and that operates to rotate along with the at least one encoder or sensor (and/or components thereof) 33, 36 the position of which is shown diagrammatically by the dashed box in FIG. 7, the probe 112, etc.) may extend from the other side of the RJ 106 through the at least one encoder or sensor 33, 36 to the probe 112. In one or more embodiments of the probe 112, the white light beam is incident on a spacer 111 via a gradient-index lens (hereinafter, GRIN lens) 109. A diffraction grating (hereinafter, diffractive element) 107 is provided at the leading end portion of the spacer in (e.g., the GRIN lens 109 and the diffraction grating 107 are located on opposite sides of the spacer iii), and as the white light beam is incident on this diffractive element 107, a spectral sequence 114 is formed on a target (e.g., an object, a specimen, a subject, a patient, etc.) 116. In one or more embodiments, the probe 112 may not include the spacer in, and the GRIN lens 109 may be connected to the diffractive element 107 to permit the spectral sequence 114 to be formed on the target 116. Reflected light from the spectral sequence 114 (e.g., light from the spectral sequence 114 that is formed on, and is reflected by, the target 116; light that is reflected by the target 116; etc.) is taken in by a detection fiber or cable 118. Although one detection fiber 118 is illustrated in FIG. 7, a plurality of detection fibers may be used. In one or more embodiments, the detection fiber 118 may extend to and/or near the end of the probe 112 (e.g., at the distal end 115 of the probe 112). For example, in the system 100 of FIG. 7, the detection fiber 118 may have a detection fiber portion (see fiber 118 extending through the probe 112 in FIG. 7) that extends from or through the RJ 106 through, and to and/or near (e.g., adjacent to the end 115 of the probe 112, about the end 115 of the probe 112, near the end 115 of the probe 112 closest to the target 116, etc.) the end of, the probe 112. The light taken in by the detection fiber 118 is separated into spectral components and detected by at least one detector, such as, but not limited to, a spectrometer 120 (and/or one or more components thereof as discussed herein), provided at the exit side of the detection fiber 118. In one or more embodiments, the end of the detection fiber 118 that takes in the reflected light may be disposed on or located near at least one of: the diffraction grating 107, the end of the spacer in, the end 115 of the probe 112, etc. Additionally or alternatively, the reflected light may be passed at least one of: through the probe 112, through the GRIN lens 109, through the rotary junction 106, etc., and the reflected light may be passed, via a deflecting or deflected section 117 (discussed below), to the spectrometer 120. As shown in FIG. 7, as the portion extending from the RJ 106 to the probe 112 is rotated about the rotational axis extending in the longitudinal direction of the probe 112, the spectral sequence 114 moves in a direction orthogonal to the spectral sequence 114, and reflectance information in two-dimensional directions may be obtained. Arraying these pieces (e.g., the reflectance information in two-dimensional directions) of information makes it possible to obtain a two-dimensional image. In one or more embodiments, the Rotary Encoder or Sensor may be used with, or in place of, the Rotary Junction 106.

Preferably, in one or more embodiments including the deflecting or deflected section 117 (best seen in FIG. 7), the deflected section 117 operates to deflect the light from the light source 101 to the probe 112, and then send light received from the probe 112 towards at least one detector (e.g., the spectrometer 120, one or more components of the spectrometer, another type of detector, etc.). In one or more embodiments, the deflected section (e.g., the deflected section 117 of the system 100 as shown in FIG. 7 may include or may comprise one or more interferometers or optical interference systems that operate as described herein, including, but not limited to, a circulator, a beam splitter, an isolator, a coupler (e.g., fusion fiber coupler), a partially severed mirror with holes therein, a partially severed mirror with a tap, etc. In one or more embodiments, the interferometer or the optical interference system may include one or more components of the system 100 (or any other system discussed herein) such as, but not limited to, one or more of the light source 101, the deflected section 117, the rotary junction 106, the Rotary Encoder or Sensor 33, 36, and/or the probe 112 (and/or one or more components thereof).

While not limited to such arrangements, configurations, devices or systems, one or more embodiments of the methods discussed herein may be used with a SEE probe as aforementioned, such as, but not limited to, for example, the system 100 (see FIG. 7), etc. In one or more embodiments, one user may perform the method(s) discussed herein. In one or more embodiments, one or more users may perform the method(s) discussed herein.

The devices and/or systems, such as, but not limited to, the system 2, the system 10, the system 100, etc.), etc., may include or be connected to a broadband light source 101 (best shown in FIG. 7 for the system 100). The broadband light source 101 may include a plurality of light sources or may be a single light source. The broadband light source 101 may include one or more of a laser, an organic light emitting diode (OLED), a light emitting diode (LED), a halogen lamp, an incandescent lamp, supercontinuum light source pumped by a laser, and/or a fluorescent lamp. The broadband light source 101 may be any light source that provides light which may then be dispersed to provide light which is then used to for spectral encoding of spatial information. The broadband light source 101 may be fiber coupled or may be free space coupled to the other components of the apparatus and/or system 100 or any other embodiment discussed herein.

As best seen in FIG. 7, the system 100 (or any other apparatus or system discussed herein) may include a rotary junction 106 (see e.g., the systems discussed herein, including, but not limited to, the system 2, the system 10, the systems of FIGS. 1-7 or any other figure discussed herein, etc.). The connection between the light source 101 and the rotary junction 106 (and/or the Rotary Encoder or Sensor 33, 36) may be a free space coupling or a fiber coupling via fiber 104 and/or waveguide/fiber 108. The rotary junction 106 (and/or the Rotary Encoder or Sensor 33, 36) may supply just illumination light via the rotary coupling or may supply one or more of illumination light, power, and/or sensory signal lines. In one or more embodiments, the driving motor M may be located at or disposed with the RJ 106, and the motor, RJ 106, and the light source 101 may be disposed inside of a console or system. A drive cable 22 (e.g., a long drive cable) may be placed in between the RJ 106/motor M and the encoder or sensor (e.g., the encoder or sensor 33, 36; the encoder or sensor 20; etc.). In one or more embodiments, the motor M and the drive cable 22 may be located in between the RJ 106 and the encoder (e.g., the encoder or sensor 33, 36; the encoder or sensor 20; etc.).

As best seen in FIG. 7, the rotary junction 106 may couple the light to a first waveguide 108. In at least one embodiment, the first waveguide 108 is a single mode fiber, a multimode fiber, or a polarization maintaining fiber. As shown in FIGS. 1-4B, the Rotary Encoder or Sensor (e.g., the encoder and/or the sensor 33, 36 shown diagrammatically in FIG. 7) may rotate or spin with the Drive Cable(s)-1 (reference number 22 in FIG. 1, for example), -2 (reference number 24 in FIG. 1, for example), and/or to the probe (e.g., the embodiment example of the probe 112 as discussed herein).

In one or more embodiments, the first waveguide 108 may be coupled to an optical apparatus and/or system that operates as an imager or imaging device, such as, for example the probe 112 (also referred to herein as an imager, imaging device or system, and/or optical apparatus and/or system). The optical apparatus and/or system (or the imager), or the probe 112, may include one or more optical components, that refract, reflect, and disperse the light from the first waveguide 108 to form at least one line of illumination light 114 (e.g., additionally or alternatively, in one or more embodiments, an imaging device or probe 112 in an apparatus or system (e.g., a SEE system, an OCT system, an IVUS system, etc.) may form a plurality of illumination lines, such as, but not limited to, from three (3) wavelength ranges in a spectrum (such as, but not limited to, in the following colors: Red (R), Green (G), Blue (B), etc.), and may overlap the plurality of illumination lines (e.g., the three (3) illumination lines) in the same or substantially the same position on the target, the object, the sample or the patient 116) on a sample, an object or a patient 116 (e.g., a predetermined area in the patient, a predetermined area in and/or on a target, through the patient, through the target, etc.). In an embodiment, the line of illumination light 114 is a line connecting focal points for a wavelength range as the illumination light exits the optical apparatus and/or system (or the imager, the imaging device, or the probe) 112, the wavelength range being determined by the light source 101. In another embodiment, the spectrometer 120 may further limit the wavelength range by only using information from specified wavelengths of interest. In another embodiment, the line of illumination light 114 is a line formed by the illumination light as the illumination light intersects a surface of the target, the sample, the object or the patient 116 for the range of wavelengths that are detected by the spectrometer 120. In another embodiment, the line of illumination light 114 is a line of illumination light in a wavelength range formed on a specific image plane which is determined by the detection optics. In one or more embodiments, only some of the points on the image line may be in focus while other points on the image line may not be in focus. The line of illumination light 114 may be straight or curved.

In an alternative embodiment, the optical apparatus and/or system (or the imager or imaging device) 112 may partially collimate the light from the waveguide 108 such that the light is focused onto the sample, the object or the patient 116 but the light is substantially collimated at a dispersive optical element such as a grating.

The apparatus (such as the system 2, 10, 100, any other apparatus/system discussed herein, etc.) may include a detection waveguide 118. The detection waveguide 118 may be a multimode fiber, a plurality of multimode fibers, a fiber bundle, a fiber taper, or some other waveguide. In one or more embodiments, preferably the detection waveguide 118 comprises a plurality of detection fibers (e.g., forty-five (45) fibers, sixty (60) fibers, in a range of 45-60 fibers, less than 45 fibers, more than 6o fibers, etc.). The plurality of detection fibers of the detection waveguide 118 may be spaced apart and located around the periphery (e.g., inside the periphery, around a border of the periphery, etc.) of the imaging device or the probe 112. The detection waveguide 118 gathers light from the target, the sample, the object and/or the patient 116 which has been illuminated by light from the optical apparatus and/or system (or the imager or the imaging device, or the probe) 112. The light gathered by the detection waveguide 118 may be reflected light, scattered light, and/or fluorescent light. In one embodiment, the detection waveguide 118 may be placed before or after a dispersive element of the optical apparatus and/or system, or the probe, 112. In one embodiment, the detection waveguide 118 may be covered by the dispersive element of the optical apparatus and/or system, or the probe, 112, in which case the dispersive element may act as wavelength-angular filter. In another embodiment, the detection waveguide 118 is not covered by the dispersive element of the optical apparatus and/or system, imager or imaging device 112. The detection waveguide 118 guides detection light from the target, the sample, the object and/or the patient 116 to the spectrometer 120.

The spectrometer 120 may include one or more optical components that disperse light and guide the detection light from the detection waveguide 118 to one or more detectors. The one or more detectors may be a linear array, a charge-coupled device (CCD), a plurality of photodiodes or some other method of converting the light into an electrical signal. The spectrometer 120 may include one or more dispersive components such as prisms, a prisms, gratings, or grisms. The spectrometer 120 may include optics and opto-electronic components which allow the spectrometer 120 to measure the intensity and wavelength of the detection light from the target, the sample, the object and/or the patient 116. The spectrometer 120 may include an analog to digital converter (ADC). The separated illumination lights (e.g., illumination light 114) are emitted from a surface of the diffraction grating 107 to illuminate the object, and reflected lights (returned lights) from the object pass through the diffraction grating 107 again and are delivered to the spectrometer 120 by the detection fiber (DF) 118. In some embodiments, the reflected lights (returned lights) from the object (e.g., the object 116) are delivered to the spectrometer 120 by the detection fiber (DF) 118 without first passing through the diffraction grating 107.

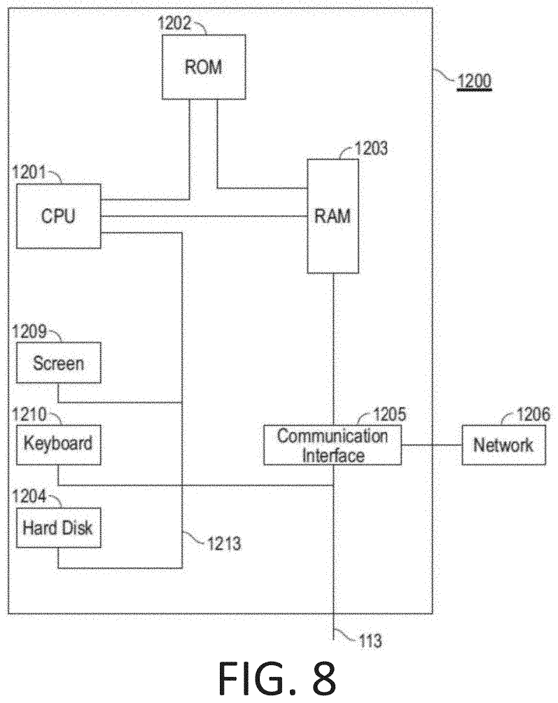

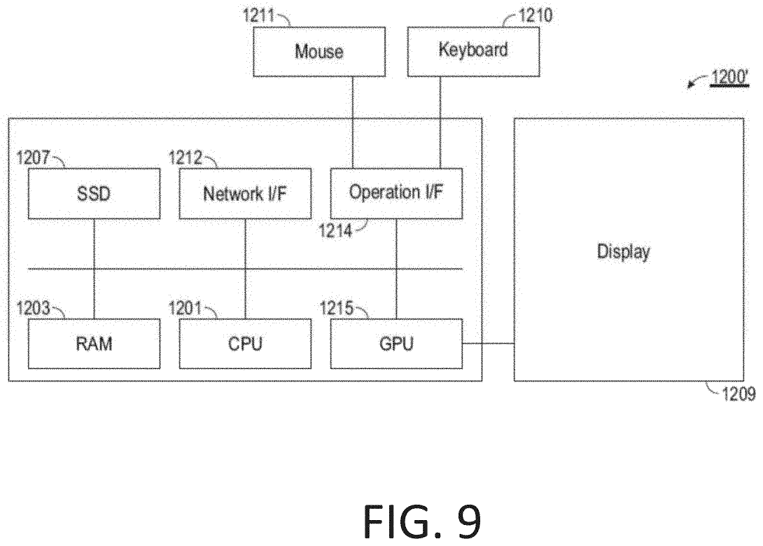

The spectrometer 120 may transmit the digital or analog signals to a processor or a computer such as, but not limited to, an image processor, a processor or computer 1200, 1200′ (see e.g., FIGS. 7 and 8-9), a combination thereof, etc. The image processor may be a dedicated image processor or a general purpose processor that is configured to process images. In at least one embodiment, the computer 1200, 1200′ may be used in place of, or in addition to, the image processor. In an alternative embodiment, the image processor may include an ADC and receive analog signals from the spectrometer 120. The image processor may include one or more of a CPU, DSP, FPGA, ASIC, or some other processing circuitry. The image processor may include memory for storing image, data, and instructions. The image processor may generate one or more images based on the information provided by the spectrometer 120. A computer or processor discussed herein, such as, but not limited to, the system 2, the computer 1200, the computer 1200′, the image processor, may also include one or more components further discussed herein below (see e.g., FIGS. 8-9).

One or more components of the apparatus and/or system (such as the system 2, 10, 100, etc.) may be rotated via the rotary junction 106 (and/or the at least one encoder or sensor 33, 36), or oscillated so as to scan a line of illumination light 114 so as to create a 2D array of illumination light. A 2D image may be formed by scanning a spectrally encoded line from the optical apparatus and/or system, the imager or imaging device, or the probe, 112 across the target, the sample, the object and/or the patient 116. The apparatus and/or system (such as the system 2, 10, 100, etc.) may include an additional rotary junction that couples the light from the detection fiber 118 to the spectrometer 120. Alternatively, the spectrometer 120 or a portion of the spectrometer 120 may rotate with the fiber 118. In an alternative embodiment, there is no rotary junction 106 and the light source rotates with the fiber 108. An alternative embodiment may include an optical component (mirror) after a dispersive element in the optical system or imager, or the probe, 112 which rotates or scans the spectrally encoded line of illumination light across the target, the sample, the object and/or the patient 116 substantially perpendicular to the spectrally encoded line of illumination light 114 in a linear line to produce a 2D image or circumferentially in a circle so as to produce a toroidal image. Substantially, in the context of one or more embodiments of the present disclosure, means within the alignment and/or detection tolerances of the apparatus and/or system (such as the system 100 or any other system discussed herein) and/or any other system being discussed herein may be utilized or accounted for. In an alternative embodiment, there is no rotary junction 106 and an illumination end of the optical apparatus and/or system or the imager, or the probe, 112 is scanned or oscillated in a direction perpendicular to the illumination line. The at least one encoder or sensor 33, 36 may be positioned between the RJ 106 (or in place of the RJ 106) and the probe 112 as shown diagrammatically in at least FIG. 7.

In one or more alternative embodiments, a dispersive element 107 (i.e., a diffraction grating) may be used in the optical apparatus and/or system, or the probe, 112 as shown, respectively, in FIG. 7. In one or more embodiments (best seen in FIG. 7), light that has been emitted from the core of the end portion of the illumination optical fiber or the first waveguide 108 may enter a spacer 111 via a refractive-index distribution lens (hereinafter referred to as “gradient index (GRIN) lens”) 109. The diffraction grating 107 is formed at the tip portion of the spacer 111 as shown in FIG. 7, and a spectral sequence 114 is formed on the target, the subject, object or sample 116 by a light flux (e.g., of white light) entering the diffraction grating 107. FIG. 7 illustrates an embodiment of apparatus and/or system 100 including a spectrometer, and a deflecting or deflected section 117 such that the cable or fiber 104 and/or the cable or fiber 108 connecting the light source 101 to the rotary junction 106 (and/or the encoder and/or the sensor 33, 36) and/or the optical apparatus and/or system (or the probe) 112 and the cable or fiber 118 connecting the spectrometer 120 to the rotary junction 106 (and/or the encoder and/or the sensor 33, 36) and/or the optical apparatus and/or system or imager (or the probe) 112 pass through, and are connected via, the deflected section 117 (discussed further below).

In at least one embodiment, a console or computer 1200, 1200′ operates to control motions of the RJ 106 (and/or the encoder and/or the sensor 33, 36) via a Motion Control Unit (MCU) or a motor 140, acquires intensity data from the detector(s) in the spectrometer 120, and displays the scanned image (e.g., on a monitor or screen such as a display, screen or monitor 1209 as shown in the console or computer 2, 1200 of any of FIGS. 1-3, 6-7 and FIG. 8 and/or the console 1200′ of FIG. 9 as further discussed below). In one or more embodiments, the MCU or the motor 140 operates to change a speed of a motor of the RJ 106 and/or of the RJ 106 (and/or the encoder and/or the sensor 33, 36). The motor may be a stepping or a DC servo motor to control the speed and increase position accuracy. In one or more embodiments, the deflection or deflected section 117 may be at least one of: a component that operates to deflect the light from the light source to the interference optical system, and then send light received from the interference optical system towards the at least one detector; a deflection or deflected section that includes at least one of: one or more interferometers, a circulator, a beam splitter, an isolator, a coupler, a fusion fiber coupler, a partially severed mirror with holes therein, and a partially severed mirror with a tap; etc. In one or more other embodiments, the rotary junction 106 may be at least one of: a contact rotary junction, a lenseless rotary junction, a lens-based rotary junction, or other rotary junction known to those skilled in the art. The rotary junction may be a one channel rotary junction or a two channel rotary junction. In one or more embodiments, the illumination portion of the SEE probe may be separate from the detection portion of the SEE probe. For example, in one or more applications, a probe may refer to the illumination assembly, which includes the illumination fiber 108 (e.g., single mode fiber, a GRIN lens, a spacer and the grating on the polished surface of the spacer, etc.). In one or more embodiments, a scope may refer to the illumination portion which, for example, may be enclosed and protected by a drive cable, a sheath, and detection fibers (e.g., multimode fibers (MMFs)) around the sheath. Grating coverage is optional on the detection fibers (e.g., MMFs) for one or more applications. The illumination portion may be connected to a rotary joint and may be rotating continuously at video rate. In one or more embodiments, the detection portion may include one or more of: the detection fiber 118, the spectrometer 120, the computer 1200, the computer 1200′, etc. The detection fibers, such as the detection fiber(s) 118, may surround the illumination fiber, such as the IF 108, and the detection fibers may or may not be covered by the grating, such as the grating 107.

In an embodiment, the first waveguide 108 may be single mode fiber. In an alternative embodiment, the first waveguide 108 may be a multimode fiber or a double clad fiber. In an embodiment, the second waveguide 118 may be a multi-mode fiber a single mode fiber, or a fiber bundle.

In an alternative embodiment, the first waveguide 108 may be an inner core of a double-clad fiber, while the second waveguide 118 may be between the inner core and the outer cladding of the double clad fiber. If a double clad fiber is used, an alternative embodiment may include an optical coupler for guiding illumination light to the inner core, and the optical coupler may also receive detection light from the outer waveguide which is then guided to the spectrometer 120.

In one or more embodiments, a SEE probe may include the illumination fiber(s) 104 and/or 108, the diffraction grating 107 and the detection fiber 118, and the illumination fiber(s) 104 and/or 108, the diffraction grating 107 and the detection fiber 118 may be housed by a metal or plastic tube to enhance the SEE probe's robustness for rotational motions and external stress by insertion. The SEE probe may further include a lens at the distal end of the probe, which may be located after the diffraction grating 107 (not shown), or between the diffraction grating 107 and the illumination fiber 108 (see e.g., the lens or prism 109 as shown in FIG. 7 and as discussed further below), or between the diffraction grating 107 and the detection fiber 118. In one or more embodiments, a SEE probe is incorporated with the motor or MCU 140 at a proximal side, which enables the SEE probe to scan in a horizontal direction, for example, with a periodical arc motion. In one or more embodiments, the motor 140 may be a rotational motor to achieve, for example, circumferential viewing. In some embodiments, the systems 2, 10, 100, or any other system discussed herein, may include one or more rotary junctions (not shown) (and/or one or more encoders and/or sensors) that are configured to rotate the illumination fiber 108 or the illumination fiber 108 and the detection fiber 118. In at least one embodiment, the detection fiber 118 may be coupled with the spectrometer 120 including a diffraction grating and the at least one detector of the spectrometer 120. In one or more embodiments one or more of the Drive Cable -1 and the Drive Cable -2 may include one or more of the illumination fiber(s) and/or the detection fiber(s) discussed herein to pass light to the probe, such as, but not limited to, the probe 112.

The output of the one or more components of any of the systems discussed herein may be acquired with the at least one detector and/or the spectrometer 120, e.g., such as, but not limited to, photodiodes, Photomultiplier tube(s) (PMTs), line scan camera(s), or multi-array camera(s). Electrical analog signals obtained from the output of the system 100 and/or the spectrometer thereof are converted to digital signals to be analyzed with a computer, such as, but not limited to, the computer 1200, 1200′. In one or more embodiments, the light source 101 may be a radiation source or a broadband light source that radiates in a broad band of wavelengths. In one or more embodiments, a Fourier analyzer including software and electronics may be used to convert the electrical analog signals into an optical spectrum. In some embodiments, the at least one detector and/or the spectrometer 120 comprises three detectors configured to detect three different bands of light. In yet other embodiments, the spectrometer 120 is configured to generate three 2D images from three different bands of light (e.g., red, green, and blue) where these three 2D images may be combined to form a single image having color information. In yet other embodiments, multiple spectrometers 120 may be used to generate different 2D images from the three different bands of light.

In accordance with at least one aspect of the present disclosure and as aforementioned, one or more methods for performing tissue characterization when using an imaging apparatus or system are provided herein. By way of at least one embodiment, a method for characterizing tissue using a SEE system may include one or more of the following: (i) setting object information; (ii) designating one or more imaging conditions; (iii) start imaging; (iv) coordinating intensities to construct a SEE image; (v) determining tissue type; (vi) displaying tissue type on a center (or other predetermined location) of a scanned tissue image; and (vii) determining whether to change a region of interest (ROI); (viii) if “Yes” the prior step, then adjusting a measuring position toward the center of the image and then determining whether to end the exam; if “No”, repeating the prior step, and if “Yes”, end the process), or if “No” in the prior step of determining whether to change the ROI, then keep displaying the scanned tissue image and tissue type and then repeat the step of determining whether to change the ROI. By way of at least another embodiment, a method may include one or more steps for performing imaging reconstruction using a real-time signal of a rotary position from a near distal end encoder. By way of at least one embodiment example, a method may include the following imaging control steps (or any other catheter methods or structure discussed in U.S. Pat. App. No. 62/634,011, filed on Feb. 22, 2018, the disclosure of which is incorporated by reference herein in its entirety, and/or any other fluid details disclosed in U.S. patent application Ser. No. 15/955,574, filed on Apr. 17, 2018, the disclosure of which is incorporated by reference herein in its entirety). For example, in at least one embodiment: A catheter sheath is inserted into a subject, such as a patient or the like. The catheter sheath may be configured as described above and may be characterized as an elongate member having proximal and distal portions. A rotatable drive cable may be disposed within the elongate member. The drive cable may be connected to a mechanism to which torque can be applied, and being mechanically coupled to an assembly. Torque is applied to the mechanism to drive the drive cable. A determination is made as to whether the applied torque exceeds a predetermined level. If the applied torque does not exceed the predetermined level, the process returns to the disengagement from the drive cable step discussed below and continues to apply torque to rotate the imaging core to image the anatomy. On the other hand, if the applied torque does exceed the predetermined level, the process proceeds to either the disengagement from the drive cable step discussed below or the withdrawal of the drive cable step discussed below. In the disengagement from the drive cable step , the mechanism is disengaged from the drive cable via sheared adhesive or broken optical fiber. Alternatively, in the withdrawal of the drive cable step, the drive cable is withdrawn from a site of complication that may occur. Should such a complication occur, upon encountering the proximal force on the rotating drive cable, this embodiment quickly disengages the distal portion including the drive cable that is inside the anatomy, promptly stopping rotation of and/or withdrawing the drive cable and/or imaging core from the site of complication to reduce the chances of a drill-through and/or perforation.

In one or more embodiments, a probe may be connected to one or more systems (e.g., the system 2, the system 10, the system 100, or any other system discussed herein, etc.) with a connection member or interface module. For example, when the connection member or interface module is a rotary junction for either a SEE probe or the aforementioned OCT system, the rotary junction may be at least one of: a contact rotary junction, a lenseless rotary junction, a lens-based rotary junction, or other rotary junction known to those skilled in the art. The rotary junction may be a one channel rotary junction or a two channel rotary junction. As aforementioned, the rotary junction (such as the RJ 106) may be used with the Rotary Encoder or Sensor 33, 36, or, in one or more alternative embodiments, the Rotary Encoder or Sensor 33, 36 may be used in place of the RJ 106.

In one or more embodiments, a SEE probe may further include a lens located between the DG 107 and the sample or object (e.g., object 116). Preferably, in such an embodiment, the lens receives light from the fiber 108, DG 107 and/or the prism 109 (depending on which system, such as the system 2, the system 10, the system 100, etc., includes the lens) and passes the light therethrough towards the sample. After illuminating the sample, the light passes through the lens back towards the DG 107 and/or the prism 109 and into the fiber 118, and/or directly into the fiber 118. In one or more embodiments, the lens may or may not be tilted or angled.

Unless otherwise discussed herein, like numerals indicate like elements. For example, while variations or differences exist between the systems, such as, but not limited to, the system 2, the system 10, the system 100, or any other system discussed herein, one or more features thereof may be the same or similar to each other, such as, but not limited to, the light source 101 or other component(s) thereof (e.g., the console 1200, the console 1200′, etc.). Those skilled in the art will appreciate that the light source 101, the motor or MCU 140, the at least one detector and/or the spectrometer 120, and/or one or more other elements of the system 100, may operate in the same or similar fashion to those like-numbered elements of one or more other systems, such as, but not limited to, the system 2, the system 10, or any other system discussed herein. Those skilled in the art will appreciate that alternative embodiments of the system 2, the system 10, the system 100, any other system discussed herein, etc., and/or one or more like-numbered elements of one of such systems, while having other variations as discussed herein, may operate in the same or similar fashion to the like-numbered elements of any of the other systems (or components thereof) discussed herein. Indeed, while certain differences exist between the system 100 of FIG. 7 and one or more embodiments shown in any of FIGS. 1-5, for example, as discussed herein, there are similarities. Likewise, while the console or computer 1200 may be used in one or more systems (e.g., the system 10, the system 100, or any other system discussed herein, etc.), one or more other consoles or computers, such as the console or computer 1200′, may be used additionally or alternatively.

There are many ways to compute intensity, viscosity, resolution (including increasing resolution of one or more images), creation of color images or any other measurement discussed herein, or to perform any imaging technique discussed herein, digital as well as analog. In at least one embodiment, a computer, such as the console or computer 1200, 1200′, may be dedicated to control and monitor the imaging (e.g., SEE, OCT, IVUS, etc.) devices, systems, methods and/or storage mediums described herein.

The electric signals used for imaging may be sent to one or more processors, such as, but not limited to, a computer 1200 (see e.g., FIG. 8), a computer 1200′ (see e.g., FIG. 9), etc. as discussed further below, via cable(s) or wire(s), such as, but not limited to, the cable(s) or wire(s) 113 (see FIG. 8).

Light emitted by a white light source may be transmitted by an illumination light transmission fiber and may be incident on a probe portion via the Rotary Encoder. Additionally or alternatively, the light emitted by the white light source may be transmitted by the illumination light transmission fiber and may be incident on the probe portion via a deflecting or deflected section and via the Rotary Encoder or Sensor (and/or a rotary junction as aforementioned).

As aforementioned, a rotary junction for a probe may be at least one of: a contact rotary junction, a lenseless rotary junction, a lens-based rotary junction, or other rotary junction known to those skilled in the art. The rotary junction may be a one channel rotary junction or a two channel rotary junction. In one or more embodiments, the illumination portion of the probe may be separate from the detection portion of the probe. For example, in one or more applications, a probe may refer to the illumination assembly, which includes an illumination fiber (e.g., single mode fiber, a GRIN lens, a spacer and the grating on the polished surface of the spacer, etc.). In one or more embodiments, a scope may refer to the illumination portion which, for example, may be enclosed and protected by a drive cable, a sheath, and detection fibers (e.g., multimode fibers (MMFs)) around the sheath. Grating coverage is optional on the detection fibers (e.g., MMFs) for one or more applications. The illumination portion may be connected to a rotary joint and may be rotating continuously at video rate. In one or more embodiments, the detection portion operating to obtain the image data may include one or more of: a detection fiber, a spectrometer, a computer 1200, the computer 1200′ (as discussed further below), etc.

Unless otherwise discussed herein, like numerals indicate like elements. For example, while the console or computer 1200 may be used in one or more systems or devices discussed herein, one or more other consoles or computers, such as the console or computer 1200′, may be used additionally or alternatively, and any like-numbered elements thereof may operate in the same or similar fashion.

There are many ways to compute intensity, viscosity, resolution (including increasing resolution of one or more images), creation of color images or any other measurement discussed herein, digital as well as analog. In at least one embodiment, a computer, such as the console or computer 1200, 1200′, may be dedicated to control and monitor the devices, systems, methods and/or storage mediums described herein.

In accordance with one or more aspects of the present disclosure, one or more methods for performing imaging may be performed using an encoder and/or a sensor as discussed herein.

Various components of a computer system 1200 are provided in FIG. 8. A computer system 1200 may include a central processing unit (“CPU”) 1201, a ROM 1202, a RAM 1203, a communication interface 1205, a hard disk (and/or other storage device) 1204, a screen (or monitor interface) 1209, a keyboard (or input interface; may also include a mouse or other input device in addition to the keyboard) 1210 and a BUS or other connection lines (e.g., connection line 1213) between one or more of the aforementioned components (e.g., including but not limited to, being connected to the console, the probe, the encoder and/or the sensor, any motor discussed herein, a light source, etc.). In addition, the computer system 1200 may comprise one or more of the aforementioned components. For example, a computer system 1200 may include a CPU 1201, a RAM 1203, an input/output (I/O) interface (such as the communication interface 1205) and a bus (which may include one or more lines 1213 as a communication system between components of the computer system 1200; in one or more embodiments, the computer system 1200 and at least the CPU 1201 thereof may communicate with the one or more aforementioned components of a device or system, such as, but not limited to, a system using an Encoder or Sensor), and one or more other computer systems 1200 may include one or more combinations of the other aforementioned components (e.g., the one or more lines 1213 of the computer 1200 may connect to other components via line 113). The CPU 1201 is configured to read and perform computer-executable instructions stored in a storage medium. The computer-executable instructions may include those for the performance of the methods and/or calculations described herein. The system 1200 may include one or more additional processors in addition to CPU 1201, and such processors, including the CPU 1201, may be used for tissue or sample characterization, diagnosis, evaluation and/or imaging. The system 1200 may further include one or more processors connected via a network connection (e.g., via network 1206). The CPU 1201 and any additional processor being used by the system 1200 may be located in the same telecom network or in different telecom networks (e.g., performing technique(s) discussed herein may be controlled remotely).

The I/O or communication interface 1205 provides communication interfaces to input and output devices, which may include a light source, a spectrometer, the communication interface of the computer 1200 may connect to other components discussed herein via line 113 (as diagrammatically shown in FIG. 8), a microphone, a communication cable and a network (either wired or wireless), a keyboard 1210, a mouse (see e.g., the mouse 1211 as shown in FIG. 9), a touch screen or screen 1209, a light pen and so on. The Monitor interface or screen 1209 provides communication interfaces thereto.

Any methods and/or data of the present disclosure, such as the methods for performing tissue or sample characterization, diagnosis, examination and/or imaging (including, but not limited to, increasing image resolution, performing imaging reconstruction using a real-time signal of a rotary position from a near distal end encoder, etc.), for example, with an encoder and/or a sensor as discussed herein, may be stored on a computer-readable storage medium. A computer-readable and/or writable storage medium used commonly, such as, but not limited to, one or more of a hard disk (e.g., the hard disk 1204, a magnetic disk, etc.), a flash memory, a CD, an optical disc (e.g., a compact disc (“CD”) a digital versatile disc (“DVD”), a Blu-ray™ disc, etc.), a magneto-optical disk, a random-access memory (“RAM”) (such as the RAM 1203), a DRAM, a read only memory (“ROM”), a storage of distributed computing systems, a memory card, or the like (e.g., other semiconductor memory, such as, but not limited to, a non-volatile memory card, a solid state drive (SSD) (see SSD 1207 in FIG. 9), SRAM, etc.), an optional combination thereof, a server/database, etc. may be used to cause a processor, such as, the processor or CPU 1201 of the aforementioned computer system 1200 to perform the steps of the methods disclosed herein. The computer-readable storage medium may be a non-transitory computer-readable medium, and/or the computer-readable medium may comprise all computer-readable media, with the sole exception being a transitory, propagating signal in one or more embodiments. The computer-readable storage medium may include media that store information for predetermined or limited or short period(s) of time and/or only in the presence of power, such as, but not limited to Random Access Memory (RAM), register memory, processor cache(s), etc. Embodiment(s) of the present disclosure may also be realized by a computer of a system or apparatus that reads out and executes computer executable instructions (e.g., one or more programs) recorded on a storage medium (which may also be referred to more fully as a “non-transitory computer-readable storage medium”) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s).

In accordance with at least one aspect of the present disclosure, the methods, systems, and computer-readable storage mediums related to the processors, such as, but not limited to, the processor of the aforementioned computer 1200, etc., as described above may be achieved utilizing suitable hardware, such as that illustrated in the figures. Functionality of one or more aspects of the present disclosure maybe achieved utilizing suitable hardware, such as that illustrated in FIG. 8. Such hardware may be implemented utilizing any of the known technologies, such as standard digital circuitry, any of the known processors that are operable to execute software and/or firmware programs, one or more programmable digital devices or systems, such as programmable read only memories (PROMs), programmable array logic devices (PALs), etc. The CPU 1201 (as shown in FIG. 8) may also include and/or be made of one or more microprocessors, nanoprocessors, one or more graphics processing units (“GPUs”; also called a visual processing unit (“VPU”)), one or more Field Programmable Gate Arrays (“FPGAs”), or other types of processing components (e.g., application specific integrated circuit(s) (ASIC)). Still further, the various aspects of the present disclosure may be implemented by way of software and/or firmware program(s) that may be stored on suitable storage medium (e.g., computer-readable storage medium, hard drive, etc.) or media (such as floppy disk(s), memory chip(s), etc.) for transportability and/or distribution. The computer may include a network of separate computers or separate processors to read out and execute the computer executable instructions. The computer executable instructions may be provided to the computer, for example, from a network or the storage medium.

As aforementioned, hardware structure of an alternative embodiment of a computer or console 1200′ is shown in FIG. 9. The computer 1200′ includes a central processing unit (CPU) 1201, a graphical processing unit (GPU) 1215, a random access memory (RAM) 1203, a network interface device 1212, an operation interface 1214 such as a universal serial bus (USB) and a memory such as a hard disk drive or a solid state drive (SSD) 1207. Preferably, the computer or console 1200′ includes a display 1209. The computer 1200′ may connect with a motor, a console, an encoder and/or a sensor or any other component of the device(s) or system(s) discussed herein via the operation interface 1214 or the network interface 1212 (e.g., via a cable or fiber, such as the cable or fiber 113 as similarly shown in FIG. 8). A computer, such as the computer 1200′, may include a motor or motion control unit (MCU) in one or more embodiments. The operation interface 1214 is connected with an operation unit such as a mouse device 1211, a keyboard 1210 or a touch panel device. The computer 1200′ may include two or more of each component.

At least one computer program is stored in the SSD 1207, and the CPU 1201 loads the at least one program onto the RAM 1203, and executes the instructions in the at least one program to perform one or more processes described herein, as well as the basic input, output, calculation, memory writing and memory reading processes.

The computer, such as the computer 1200, 1200′, may communicate with an MCU, an encoder and/or a sensor, etc. to perform imaging, and reconstructs an image from the acquired intensity data. The monitor or display 1209 displays the reconstructed image, and may display other information about the imaging condition or about an object to be imaged. The monitor 1209 also provides a graphical user interface for a user to operate any system discussed herein. An operation signal is input from the operation unit (e.g., such as, but not limited to, a mouse device 1211, a keyboard 1210, a touch panel device, etc.) into the operation interface 1214 in the computer 1200′, and corresponding to the operation signal the computer 1200′ instructs any system discussed herein to set or change the imaging condition (e.g., improving resolution of an image or images), and to start or end the imaging. A light or laser source and a spectrometer and/or detector may have interfaces to communicate with the computers 1200, 1200′ to send and receive the status information and the control signals.

The present disclosure and/or one or more components of devices, systems and storage mediums, and/or methods, thereof also may be used in conjunction with any suitable optical assembly including, but not limited to, SEE probe technology, such as in U.S Pat. Nos. 6,341,036; 7,447,408; 7,551,293; 7,796,270; 7,859,679; 8,045,177; 8,145,018; 8,838,213; 9,254,089; 9,295,391; 9,415,550; 9,557,154 and arrangements and methods of facilitating photoluminescence imaging, such as those disclosed in U.S. Pat. No. 7,889,348 to Tearney et al. Other exemplary SEE systems are described, for example, U.S. Pat. Pubs. 2016/0349417; US2017/0035281; 2017/167861; 2017/0168232; 2017/0176736; 2017/0290492; 2017/0322079; and WO2015/116951; WO2015/116939; WO2017/117203; WO2017/024145; WO2017/165511A1 and U.S. Non-Provisional patent application Ser. No. 15/418,329 filed Jan. 27, 2017 each of which patents, patent publications and application(s) are incorporated by reference herein in their entireties.

Similarly, the present disclosure and/or one or more components of devices, systems and storage mediums, and/or methods, thereof also may be used in conjunction with optical coherence tomography probes. Such probes include, but are not limited to, the OCT imaging systems disclosed in U.S. Pat. Nos. 7,872,759; 8,289,522; 8,676,013; 8,928,889; 9,557,154 and U.S. Pat. Pub. 2017/0135584; and WO 2016/015052 to Tearney et al, as well as the disclosures directed to multimodality imaging disclosed in U.S. Pat. No. 9,332,942, and U.S. Patent Publication Nos. 2010/0092389, 2011/0292400, 2012/0101374 and 2016/0228097, and WO 2016/144878 each of which patents and patent publications are incorporated by reference herein in their entireties.

Similarly, the present disclosure and/or one or more components of devices, systems and storage mediums, and/or methods, thereof also may be used in conjunction with imaging catheter technologies and methods, such as, but not limited to, apparatuses, assemblies, systems, methods and/or storage mediums disclosed in at least, but not limited to: U.S. Pat. App. No. 62/634,011, filed on Feb. 22, 2018, the disclosure of which is incorporated by reference herein in its entirety, and/or any other details disclosed in U.S. patent application Ser. No. 15/955,574, filed on Apr. 17, 2018, the disclosure of which is incorporated by reference herein in its entirety.

Although the disclosure herein has been described with reference to particular embodiments, it is to be understood that these embodiments are merely illustrative of the principles and applications of the present disclosure (and are not limited thereto), and the invention is not limited to the disclosed embodiments. It is therefore to be understood that numerous modifications may be made to the illustrative embodiments and that other arrangements may be devised without departing from the spirit and scope of the present disclosure. The scope of the following claims is to be accorded the broadest interpretation so as to encompass all such modifications and equivalent structures and functions.

What is claimed is:

1. An imaging system comprising:
a probe having a proximal end and a distal end operating to communicate a probing signal with a specimen, object, or target;
at least one drive cable; and
a rotary encoder or sensor in communication with, or attached to, the at least one drive cable, the probe being positioned on a first side or a distal side of the rotary encoder or sensor,
wherein the probe, the at least one drive cable, and the rotary encoder or sensor rotate using a drive, and
wherein a first portion of, or a first drive cable of, the at least one drive cable is positioned on a second side or a proximal side of the rotary encoder or sensor, and has a length such that non-uniform rotational distortion (NURD) distortion(s) is/are reduced, minimized, or eliminated from the image of the specimen, object, or target.

2. The system of claim 1, wherein the rotary encoder or sensor of the probe one or more of:
(i) includes the at least one drive cable in or through the rotary encoder or sensor;
(ii) is disposed between the first portion of, or the first drive cable, of the at least one drive cable and a second portion of, or a second drive cable of, the at least one drive cable;
(iii) is disposed such that the a first portion of, or a first drive cable of, the at least one drive cable is positioned on the second side or the proximal side of the rotary encoder or sensor and the second portion of, or the second drive cable of, the at least one drive cable is located on the first side or the distal side of the rotary encoder or sensor;
(iv) is disposed such that the a first portion of, or a first drive cable of, the at least one drive cable is positioned on the second side or the proximal side of the rotary encoder or sensor and the second portion of, or the second drive cable of, the at least one drive cable is located on the first side or the distal side of the rotary encoder or sensor such that the second portion of, or the second drive cable of, the at least one drive cable is located between the rotary encoder or sensor and the probe;
(v) is disposed on one end of the second portion of, or the second drive cable of, the at least one drive cable and the probe is located on another end of the second portion of, or the second drive cable of, the at least one drive cable;
(vi) has the at least one drive cable extending through the rotary encoder or sensor; and/or
(vii) operates to measure an angular position of the at least one drive cable, the first portion of the at least one drive cable and the second portion of the at least one drive cable, or the first drive cable of the at least one drive cable and the second drive cable of the at least one drive cable.

3. The system of claim 2, further comprising first and second connection couplers, the first connection coupler being disposed between the first portion of, or the first drive cable of, the at least one drive cable and the rotary encoder or sensor, and the second connection coupler being disposed between the second portion of, or the second drive cable of, the at least one drive cable and the rotary encoder or sensor.

4. The system of claim 3, wherein one or more of the first drive cable or the first portion of the at least one drive cable, the second drive cable or the second portion of the at least one drive cable, and the rotary encoder or sensor operate to be disposable or reusable independently.

5. The system of claim 1, further comprising the drive that rotates the probe, the at least one drive cable, and the rotary encoder or sensor,
wherein one or more of the following:
(i) the drive is disposed such that the first portion of, or the first drive cable of, the at least one drive cable is positioned between the drive and the rotary encoder or sensor;
(ii) the length of the first portion of, or the first drive cable of, the at least one drive is the length between the drive and the rotary encoder or sensor; and/or
(iii) the drive is a proximal driving motor.

6. The system of claim 1, wherein one or more of:
(i) data or a signal from the rotary encoder or sensor comprises real-time rotary or angular position data from the rotary encoder or sensor;
(ii) the rotary encoder or sensor is used near a distal end of the probe or at a predetermined length or distance from the probe to reduce, minimize, or avoid/eliminate the NURD; and/or
(iii) the rotary encoder or sensor is used near a distal end of the probe or at a predetermined length or distance from the probe to reduce, minimize, or avoid/eliminate the NURD, where the predetermined length or distance from the probe is in a range from about one centimeter to about 50 centimeters away from the distal end of the probe.

7. The system of claim 1, further comprising a mass applied to the at least one drive cable at a location of the rotary encoder or sensor such that the mass operates as a flywheel to achieve an even rotation speed and/or to improve a rotational inertia of the rotational component(s) at the location of the mass, wherein the improved rotational inertia operates to stabilize the rotary motion and to reduce rotational speed variation(s) and the NURD.

8. The system of claim 1, wherein one or more of:
(i) the at least one drive cable comprises a single drive cable used or extending from the probe, wherein the single drive cable is disposed in and/or through a center hole or a hollow shaft of the rotary encoder or sensor and mechanically attaches to a code disk or wheel of the rotary encoder or sensor; and/or
(ii) the system further comprises the drive, the the at least one drive cable comprises the single drive cable used or extending between the drive and the probe, and the single drive cable is disposed in and/or through the center hole or the hollow shaft of the rotary encoder or sensor such that the single drive cable mechanically attaches to a code disk or wheel of the rotary encoder or sensor.

9. The system of claim 8, wherein, in a case where the single drive cable drives the probe, the rotary encoder or sensor or a portion thereof rotates together with the single drive cable.

10. The system of claim 1, further comprising at least one processor that operates to: (i) acquire the probing signal and/or data of the rotary encoder or sensor to reduce, minimize, and/or eliminate the NURD, and/or (ii) to generate or reconstruct the image having the reduced, minimized, and/or eliminated NURD.

11. The system of claim 10, wherein the at least one processor receives the probing signal and/or the data simultaneously and/or in real-time, and reduces, minimizes, or eliminates the NURD from the generated image or while generating or reconstructing the image.

12. The system of claim 1, wherein an integration portion of the probe to include the encoder or sensor integrated therein has an equal or larger outer diameter (OD) than a rest, or a remaining portion, of the probe such that sufficient space is provided for the integration of the rotary encoder or sensor.

13. The system of claim 1, further comprising one or more of:
(i) a sheath or a handle in which the probe is located; and/or
(ii) a stationary portion of the system, wherein the stationary portion comprises at least a signal source and one or more signal detector subsystems.
